(19) United States
(12) Patent Application Publication
(10) Pub. No.: US 2020/0326457 A1
(43) Pub. Date: Oct. 15, 2020

(54) LENS FOR MEDICAL DEVICE AND MANUFACTURING METHOD THEREOF

(71) Applicants: Korea Institute of Science and Technology, Seoul (KR); Korea University Research and Business Foundation, Seoul (KR)

(72) Inventors: Hojeong JEON, Seoul (KR); Jungmok SEO, Seoul (KR); Yeon Taek LEE, Seoul (KR); Seung Hoon LEE, Seoul (KR); Seung No HONG, Seoul (KR)

(21) Appl. No.: 16/838,030

(22) Filed: Apr. 2, 2020

(30) Foreign Application Priority Data
Apr. 9, 2019 (KR) 10-2019-0041628

(51) Int. Cl. G02B 1/18; C03C 15/00; C03C 23/00; C03C 17/30; A61B 1/00

(52) U.S. Cl. CPC G02B 1/18; C03C 15/00; C03C 23/0025; C03C 2217/76; A61B 1/00195; C03C 2218/111; C03C 17/30

(57) ABSTRACT

Provided is a lens for a medical device. The lens includes a microstructural layer on a surface of the lens, and a coating layer on the microstructural layer. The coating layer is made of a self-assembled monolayer (SAM). Also provided is a method of manufacturing a lens for a medical device. The method includes forming a microstructural layer on a surface of the lens; and forming a coating layer on the surface on which the microstructure is formed. The coating layer is made of a self-assembled monolayer (SAM).

### CROSS-REFERENCE TO RELATED APPLICATION

This application claims the benefit of Korean Patent Application No. 10-2019-0041628, filed on Apr. 9, 2019, in the Korean Intellectual Property Office, the disclosure of which is incorporated herein in its entirety by reference.

### BACKGROUND

### 1. Field

The present invention relates to a lens used in a medical device such as an endoscope and a manufacturing method thereof, and more particularly to, a lens for a medical device, contamination thereof caused by moisture or various foreign substances such as bodily secretions being effectively prevented by surface treatment and a manufacturing method thereof.

### 2. Description of the Related Art

Conventionally, open surgery where an incision is made on the abdomen has been widely performed. However, problems including large scars remaining on the body, a lot of blood loss, and serous pain afterward may occur. In order to solve these problems caused by conventional open surgery, various endoscopes are widely used in the medical field to perform medical examination or treatment in the interior of cavities or tubes of the body. Such an endoscope is generally used in an environment where an insert part or tip of the endoscope on which a camera is installed is easily contaminated, and an observation window of a lens installed at the tip of the endoscope needs to be kept clean at all times to obtain a clear view during a medical procedure.

When endoscopic surgery and laparoscopic surgery using an endoscope is performed, various biological substances such as tissue fluid, saliva, or blood in the human body as well as moisture often contaminate the outer surface of a transparent window. Since such contaminants obstruct examination of the interior of the body using a camera, it is inconvenient to wipe the transparent window of the tip of the camera after taking the camera out of the body during surgery or medical procedure.

Such contamination problems of the camera increase surgery time or procedure time. Particularly, in emergency situations, such as acute bleeding situations, the risk of a patient may increase during surgery or procedure. In addition, with the development of minimally invasive surgery, a diameter of an endoscope camera decreases, and thus the number of contamination tends to rapidly increase.

Also, although lenses having coating layers formed on the surfaces thereof have been conventionally used to prevent contamination caused by foreign substances, the use thereof is limited in that the lenses are available only a small number of times or for a short period of time due to very low durability of the coating layers.

### SUMMARY

Thus, the present invention has been proposed to solve various problems including the above problems, and an object of the present invention is to provide a lens for a medical device having excellent super water-repellent properties enabling efficient removal of various foreign substances or moisture contaminating the surface of the lens as well as excellent durability, and a manufacturing method thereof.

However, these problems to be solved are illustrative and the scope of the present invention is not limited thereby.

Additional aspects will be set forth in part in the description which follows and, in part, will be apparent from the description, or may be learned by practice of the presented embodiments.

According to an aspect of the present invention to achieve the object, provided is a lens for a medical device including a microstructural layer on a surface of the lens, and a coating layer on the microstructural layer.

According to an embodiment of the present invention, the coating layer may be made of a self-assembled monolayer (SAM).

According to an embodiment of the present invention, the coating layer may include at least one selected from (heptadecafluoro-1,1,2,2,-tetrahydrodecyl)trichlorosilane (FDTS), trichloro(1H,1H,2H,2H-perfluorooctyl)silane (PFOTS), triethoxy(1H,1H,2H,2H-perfluoro-1-octyl)silane (POTS), and 1H,1H,2H,2H-perfluorodecyltrichlorosilane (FDDTS).

According to an embodiment of the present invention, the lens may further include a lubricant layer on the coating layer.

According to an embodiment of the present invention, the lubricant layer may include fluorinated synthetic oil or silicone oil.

The fluorinated synthetic oil may include perfluoropolyether (PFPE) or perfluorodecalin (PFD).

According to an embodiment of the present invention, the lubricant layer may have a refractive index of 1.3 to 1.7.

According to an embodiment of the present invention, the microstructural layer may include fine protrusions each having a size of 0.5 μm to 0.8 μm.

According to an embodiment of the present invention, the microstructural layer may include fine protrusions each having a size of 90 nm to 175 nm.

According to another aspect of the present invention, provided is a method of manufacturing a lens fora medical device, the method including: forming a microstructural layer on a surface of the lens; and forming a coating layer on the surface on which the microstructure is formed.

According to an embodiment of the present invention, the coating layer may be made of a self-assembled monolayer (SAM).

According to an embodiment of the present invention, the forming of the microstructural layer may include at least one process selected from emitting a laser beam or electron beam to the surface of the lens, chemically etching the surface of the lens, and injecting-molding using a mold having a microstructure formed on the surface.

According to an embodiment of the present invention, the microstructure formed on the surface of the mold may be formed by a laser beam.

According to an embodiment of the present invention, the forming of the microstructural layer may be performed by forming a pattern including fine protrusions by emitting a laser beam to the surface of the lens.

According to an embodiment of the present invention, the pattern may be formed by adjusting a transverse pulse overlap rate and a longitudinal pulse overlap rate of the laser beam.

According to an embodiment of the present invention, the sizes of the fine protrusions may be adjusted within the range of 0.5 μm to 0.8 μm by controlling the transverse pulse overlap rate to be equal to or more than 0% and equal to or less than 50% and the longitudinal pulse overlap rate to be equal to or more than 50% and equal to or less than 75%.

According to an embodiment of the present invention, the sizes of the fine protrusions may be adjusted within the range of 90 nm to 175 nm by controlling the transverse pulse overlap rate to be more than 50% and less than 99.9% and the longitudinal pulse overlap rate to be more than 75% and less than 99.9%.

According to an embodiment of the present invention, the method may further include applying a lubricant on the surface of the lens on which the coating layer is formed after the forming of the coating layer.

According to another aspect of the present invention, provided is a method of increasing transmittance of a lens for a medical device.

In the method of increasing transmittance of the lens, the lens includes a microstructural layer on a surface and a coating layer on the microstructural layer as a self-assembled monolayer (SAM), and the method may include applying a lubricant having a refractive index corresponding to 80% to 120% of a refractive index of the lens to the coating layer.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram of a lens for an endoscope according to an embodiment of the present invention having improved super water-repellent properties;

FIG. 2 is a flowchart illustrating a method of manufacturing a lens for an endoscope according to an embodiment of the present invention;

FIGS. 3A, 3B, 4A, 4B and 4C are scanning electron microscope (SEM) images of surfaces of lenses according to experimental examples of the present invention after surface treatment;

FIGS. 5A and 5B show images of blood spreading on surfaces of lenses according to an experimental example of the present invention and a comparative example depending on formation of a microstructural layer;

FIG. 6 is a graph illustrating contact angles of liquids with a surface of a lens according to an experimental example of the present invention depending on formation of a microstructural layer and an SAM coating layer on the surface of the lens;

FIG. 7 shows a change in contact angle of a lens according to an experimental example of the present invention by sterilization and washing;

FIGS. 8A and 8B show SEM images of lenses according to an experimental example of the present invention and a comparative example with respect to the number of taping tests;

FIGS. 9A, 9B and 9C show comparison results of transmittance of lenses according to experimental examples of the present invention and comparative examples depending on application of a lubricant thereto after forming a microstructural layer and an SAM coating layer; and

FIG. 10 is a diagram illustrating overlap rates of a laser beam having a spot radius of r.

### DETAILED DESCRIPTION

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the invention, although different, are not necessarily mutually exclusive. For example, a particular feature, structure, or characteristic described herein, in connection with one embodiment, may be implemented within other embodiments without departing from the spirit and scope of the invention. In addition, it is to be understood that the location or arrangement of individual elements within each disclosed embodiment may be modified without departing from the spirit and scope of the invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views.

Hereinafter, embodiments of the present invention will be described in detail with reference to the accompanying drawings to allow those skilled in the art to easily practice the invention.

A lens for medical devices according to an embodiment of the present invention includes a microstructural layer formed on a surface of the lens, and a coating layer formed on the microstructural layer. In addition, according to an embodiment of the present invention, the lens for medical devices may further include a lubricant layer formed on the coating layer.

Hereinafter, a configuration of a lens for medical devices will be described using an endoscope, as a representative medical device, in which the lens constitutes a part thereof.

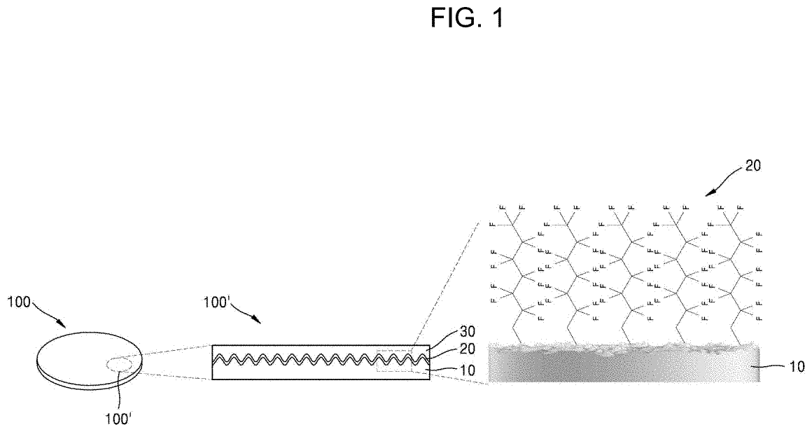

FIG. 1 is a schematic diagram of a lens 100 for an endoscope according to an embodiment of the present invention having improved super water-repellent properties.

Referring to FIG. 1 illustrating the lens 100 for an endoscope according to an embodiment of the present invention, the lens 100 for an endoscope includes a microstructural layer 10 and a coating layer 20. The microstructural layer 10 includes, for example, a fine protrusion structure having irregularities.

The lens 100 may be formed of a transparent material such as glass or a polymer plastic material. The microstructural layer 10 may be formed on the surface of the lens 100 by appropriate surface treatment.

For example, as the surface treatment, the surface of the lens may be irradiated with a laser beam or electron beam having a high energy and locally melted and solidified to form the fine protrusion structure.

Alternatively, a fine protrusion structure may be formed by local etching using an etching solution such as an acid or an etching gas having a specific radical.

As another example, when a polymer plastic material is used to form the lens, a lens having a fine protrusion structure formed on the surface thereof may be manufactured by injection molding using a mold. The mold may have a fine protrusion structure on the surface thereof. For example, the fine protrusion structure may be formed by applying a laser beam onto the surface of the mold. In this case, the surface of the mold is locally melted and solidified to form the fine protrusion structure, and the fine protrusion structure formed on the surface of the mold may be transferred to the surface of the lens during the injection molding process, thereby forming the fine protrusion structure on the surface of the lens.

Hereinafter, a lens having a microstructural layer formed by using a laser beam, will be described as a representative embodiment.

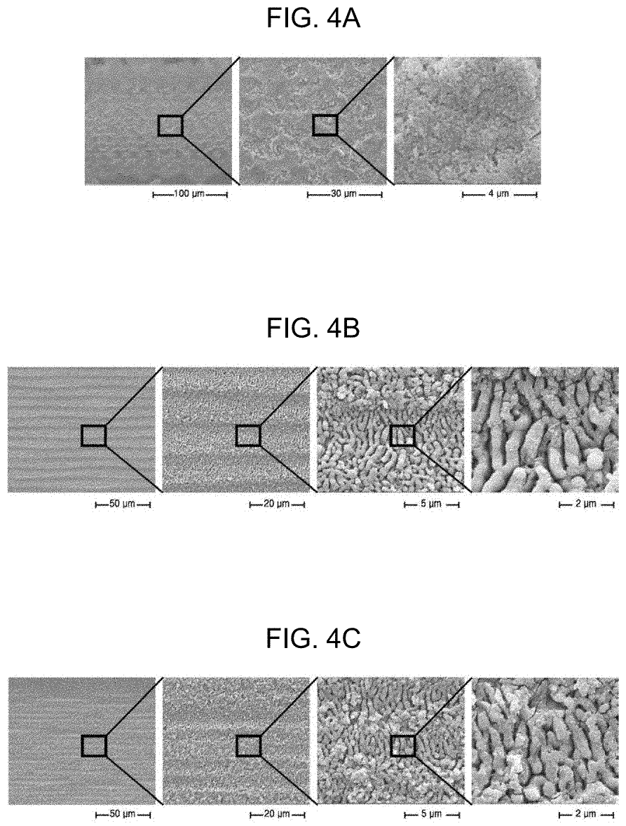

First, the microstructural layer 10 may be a layer having a pattern formed by applying a laser beam onto the surface of the lens. The pattern may have micrometer-sized fine protrusions. Patterns having micrometer-sized fine protrusions are shown in FIG. 4B. Referring to the patterns, each of the fine protrusions may have an oval shape with a major axis and a minor axis when observed above the fine protrusions. In this regard, the size of each fine protrusion may refer to a length of the minor axis, and the size of the fine protrusion may be in the range of 0.5 μm to 0.8 μm.

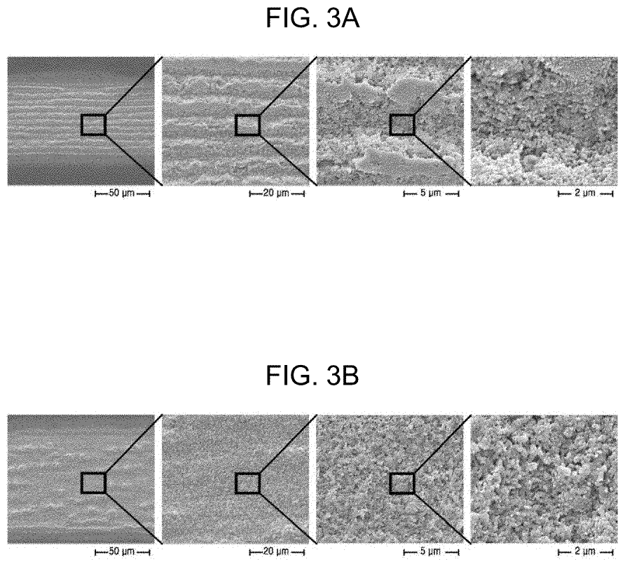

Also, the pattern may have nanometer-sized fine protrusions. Patterns having nanometer-sized fine protrusions are shown in FIG. 3A. Referring to the patterns, each of the fine protrusions may have an approximately spherical shape when observed above the fine protrusions. In this regard, the size of each fine protrusion may refer to a diameter of the fine protrusion and the size of the fine protrusion may be in the range of 90 nm to 175 nm.

Since the microstructural layer 10 having a micrometer-scale or nanometer-scale roughness is formed as described above, an air pocket is formed between the surface of the lens 100 and a liquid. Thus, an area of the surface of the lens 100 with which the liquid is able to be in contact may be minimized, and liquid droplets do not stick to the surface but roll off the surface.

When the microstructural layer 10 is formed on the surface of the lens 100, the coating layer 20 is formed on the microstructural layer 10. The coating layer 20 may be a self-assembled monolayer (SAM).

The SAM is a monolayer assembly of organic molecules spontaneously formed on a surface of a solid and each molecule includes a head group, an alkyl chain (hydrocarbon chain), and a terminal group. The head group, as the first part, is chemically adsorbed to the surface of the solid to form a close-packed monolayer. The alkyl chain, as the second part, forms an aligned monolayer by Van der Waals interactions among long chains. The terminal group, as the last part, is a functional group, and various functional groups may be applied thereto.

The lens 100 may further include a lubricant layer 30 formed on the coating layer 20, optionally. The lubricant layer 30 applied to the surface of the lens 100, which is located at the tip of the endoscope, may not only enhance lubricity but also increase transmittance of the surface treated lens 100. In this regard, the lubricant layer 30 may also perform a function of increasing transmittance in the present invention.

To this end, a transparent material having a refractive index identical or similar to that of the lens 100 may be used to form the lubricant layer 30. The refractive index of the lubricant layer 30 may correspond to 80 to 120%, preferably 90% to 110%, of the refractive index of the lens 100. For example, when the refractive index of the lens 100 is 1.5, the refractive index of the lubricant layer 30 may be in the range of 1.3 to 1.7.

Light passing through the lens 100 is refracted by the fine protrusions formed on the surface of the lens 100, resulting in reduction in transmittance. By coating the upper surfaces of the fine protrusions with a transparent material having a refractive index identical or similar to the refractive index of the lens 100, the reduction in transmittance caused by refraction due to fine protrusions may be compensated for. Thus, transmittance may be increased.

A lubricant constituting the lubricant layer 30 may include, for example, fluorinated synthetic oil or silicone oil as a transparent organic material. The fluorinated synthetic oil may include a perfluoropolyether (PFPE)-based or perfluorodecalin (PFD)-based material. The PFPE-based lubricant may include, for example, Krytox™ GPL series (GPL103, GPL101, GPL100, or the like).

The lubricant layer 30 is adhered to the terminal group of the SAM of the coating layer 20 and strong adhesion therebetween may be maintained by Van der Waals force between —CF₃ of the terminal group of the SAM and —CF₃ of the lubricant.

The lubricant layer 30 may be applied to the coating layer 20 of the lens 100 as a fixed layer and used for a long time.

As another example, the lubricant layer 30 may be applied to the coating layer 20 for a predetermined time and then removed, and this process may be repeated. For example, an operate using the endoscope may repeat a process of applying the lubricant layer 30 to the coating layer 20 of the lens 100 before using the endoscope and removing the lubricant layer 30 after the use of the endoscope is completed.

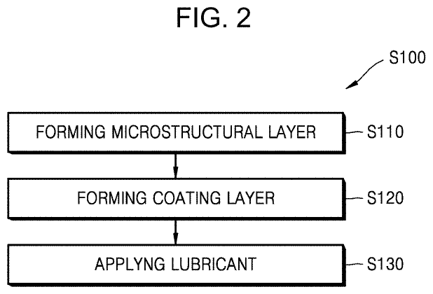

FIG. 2 is a flowchart illustrating a method of manufacturing a lens for an endoscope according to an embodiment of the present invention.

Referring to FIG. 2, a method of manufacturing a lens for a medical device (S100) may include forming a microstructural layer 10 on a surface of a lens 100 (S110), and forming a coating layer 20 (S120). Also, the method may further include applying a lubricant to the coating layer 20 (S130) after forming the coating layer, optionally.

First, the forming of the microstructural layer 10 (S110) may be performed by forming a pattern by applying a laser beam to the surface of the lens 100 using a laser generator. In this case, the laser generator may be, for example, an ytterbium nanosecond or femtosecond pulsed laser. The nanosecond pulsed layer refers to a laser having a short pulse width of 10⁻⁹ seconds with a pulse time of several nanoseconds, and the femtosecond pulsed laser refers to a laser having a very short pulse width of 10⁻¹⁵ seconds. However, the present invention is not limited thereto, and any laser capable of forming a nano-structural layer on the surface of the lens may also be used.

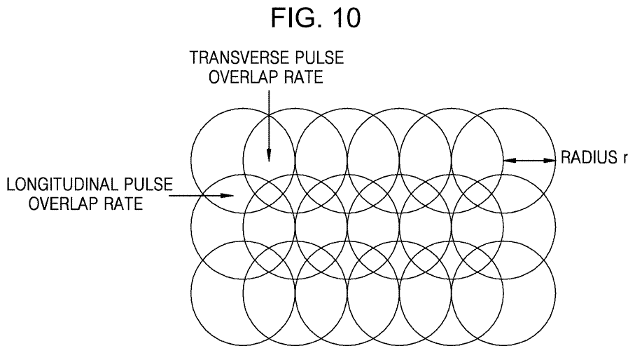

The formation of the pattern may be controlled by adjusting an overlap rate of the laser beam. The overlap rate is a ratio of overlapping area between adjacent laser beam spots. When the adjacent laser beam spots completely overlap each other, the overlap rate is 100%. The overlap rate of the laser beam is determined in consideration of spot size, pulse period, scanning speed, and the like of the laser pulse. The shape of the pattern may vary according to a difference of the overlap rate between pulses. FIG. 10 is a diagram illustrating overlap rates of a laser beam having a spot radius of r. FIG. 10 shows a transverse pulse overlap rate and a longitudinal pulse overlap rate.

In the present invention, wavelength, pulse energy, spot size, and scanning speed of the laser are constantly maintained and the transverse pulse overlap rate and the longitudinal pulse overlap rate are adjusted in order to control formation of the pattern, and thus various patterns are formed. Particularly, by adjusting the ranges of the transverse pulse overlap rate and the longitudinal pulse overlap rate, roughnesses of the patterns were adjusted from a nanometer-scale to a micrometer-scale.

For example, when the transverse pulse overlap rate is adjusted to be equal to or more than 0% and equal to or less than 50%, and the longitudinal pulse overlap rate is adjusted to be equal to or more than 50% and equal to or less than 75%, a pattern having a micrometer-scale roughness may be formed. In this regard, the pattern having a micrometer-scale roughness may include micrometer-scale protrusions. In this case, each micro-scale protrusion may have a size of 0.5 μm to 0.8 μm.

As another example, when the transverse pulse overlap rate is adjusted to be more than 50% and less than 99.9% and the longitudinal pulse overlap rate is adjusted to be more than 75% and less than 99.9%, a pattern having a nanometer-scale roughness may be formed. In this regard, the pattern having a nanometer-scale roughness may include nanometer-scale protrusions. In this case, each nanometer-scale protrusion may have a size of 90 nm to 175 nm.

Next, the forming of the coating layer 20 (S120) refers to forming the SAM on the microstructural layer 10. The SAM may be selected from (heptadecafluoro-1,1,2,2,-tetrahydrodecyl)trichlorosilane (FDTS), trichloro(1H,1H,2H,2H-perfluorooctyl)silane (PFOTS), triethoxy(1H,1H,2H,2H-perfluoro-1-octyl)silane (POTS), and 1H,1H,2H,2H-perfluorodecyltrichlorosilane (FDDTS).

FDTS, one of the materials forming the SAM, has a trichloro-silane group in the head group and is firmly fixed by covalent bonds to a surface terminated with a hydroxyl group (—OH) such as glass, ceramic, or SiO₂ which generally forms covalent bonds.

A contact angle of a liquid with the lens 100 including the coating layer 20 may be greater than 130°. A surface energy of the lens 100 decreases by formation of the microstructural layer 10, and thus the surface of the lens 100 has hydrophobicity. Since the SAM, as the coating layer 20, is formed on the surface of the microstructural layer 10, the surface energy of the lens 100 further decreases, resulting in an increase in a contact angle of a liquid with the surface of the lens 100.

The method may further include applying a lubricant (S130), optionally, after forming the coating layer 20. As described above, the applying of the lubricant (S130) may be performed for the purpose of not only enhancing lubricity but also increasing transmittance of the lens on which the coating layer 20 is formed.

The lubricant layer 30 formed by applying the lubricant may be a fixed layer formed on the coating layer 20 and used for a long time. In this case, the lubricant layer 30 may be formed by applying the lubricant to the coating layer 20 by various coating methods such as dry coating or wet coating using the lens 100 on which the coating layer 20 is formed as a target of coating.

As another example, the lubricant layer 30 may be a consumable used only once during the use of the endoscope and then disposed. In this case, the applying of the lubricant (S130) may be performed by an operator of the endoscope. For example, the operator of the endoscope may repeat a process of applying a viscous ointment-type lubricant to the coating layer 20 of the lens 100 immediately before using the endoscope, using the endoscope, and removing the lubricant after the use of the endoscope is completed.

Since transmittance of the lens increases by applying the lubricant thereto, a clear view may be stably obtained.

Hereinafter, the present invention will be described in more detail with reference to the following examples. However, these examples are for illustrative purposes only, and the present invention is not intended to be limited by these examples.

1. Formation of Microstructural Layer on Surface of Lens

Lenses were prepared for experiments. A glass lens commonly used in endoscopes was used. A microstructural layer was formed on the surface of each lens by using a laser beam. In this regard, roughness of the surface was controlled by adjusting a transverse pulse overlap rate and a longitudinal pulse overlap rate while a wavelength of 343 nm, a pulse energy of 155±10 μJ, a spot size of 20 μm, and a scanning speed of 10 mm/s were constantly maintained. Here, lenses having roughnesses varied according to the transverse pulse overlap rate and the longitudinal pulse overlap rate are referred to as Experimental Examples 1 to 5. Also, a lens that is not surface-treated is referred to as Comparative Example 1.

TABLE 1

| | Transverse pulse | Longitudinal pulse | Surface |
|---|---|---|---|
| No. | overlap rate (%) | overlap rate (%) | roughness |
| Experimental | 96.8 | 96 | Nano |
| Example 1 | | | |
| Experimental | 96.8 | 92.8 | Nano |
| Example 2 | | | |
| Experimental | 0 | 50 | Micro |
| Example 3 | | | |
| Experimental | 50 | 50 | Micro |
| Example 4 | | | |
| Experimental | 50 | 75 | Micro |
| Example 5 | | | |
| Comparative | X | X | — |
| Example 1 | | | |

FIGS. 3A, 3B, 4A, 4B and 4C are scanning electron microscope (SEM) images of surfaces of lenses according to experimental examples of the present invention after surface treatment.

First, FIGS. 3A and 3B are SEM images of lenses on which a microstructural layer is formed by adjusting the transverse pulse overlap rate and the longitudinal pulse overlap rate according to Experimental Examples 1 and 2, respectively. In FIGS. 3A and 3B, each right image is an enlarged view of a box in each left image.

Referring to FIGS. 3A and 3B, it was confirmed that patterns were formed in a transversal direction that is the scanning direction of the laser beam. As a result of observing enlarged views, it was confirmed that nanometer-scale surface roughnesses having an average protrusion size of 200 nm or less were obtained in Experimental Examples 1 and 2. Specifically, in the case of Experimental Example 1, the sizes of protrusions were 135±40 nm.

FIGS. 4A, 4B and 4C are SEM images of lenses on which a microstructural layer is formed under the conditions according to Experimental Examples 3, 4, and 5, respectively. Referring to the images, it was also confirmed that patterns were formed in the transversal direction. As a result of observing enlarged views, it was confirmed that micrometer-scale surface roughnesses having an average protrusion size of 0.5 μm or more were obtained in all experimental examples. Specifically, in the case of Experimental Example 4, the sizes of the protrusions were 0.65±0.127 μm.

Based on FIGS. 3A to 4C, it was confirmed that the surface had more uniform nanometer-scale roughness as the longitudinal pulse overlap rate increased.

2. Formation of Self-Assembled Monolayer (SAM) Coating Layer

A self-assembled monolayer (SAM) coating layer was formed on the lens on which the microstructural layer was formed by liquid phase deposition. 40 ml of toluene in which a molecular sieve (4×10⁻¹⁰ m) was dipped for 3 days or more and 400 μl of FDTS as a material used to form the SAM were mixed in a falcon tube. Then, the lens according to Experimental Example 1 on which the microstructural layer was formed was dipped in a solution contained in the falcon tube and incubated for 24 hours to form a coating layer. The resulting lens obtained thereby is referred to as Experimental Example 6. Also, the lens according to Experimental Example 1 was coated with ultra-ever dry that is a commercially available water-repellent coating agent, and the resulting lens obtained thereby is referred to as Comparative Example 2.

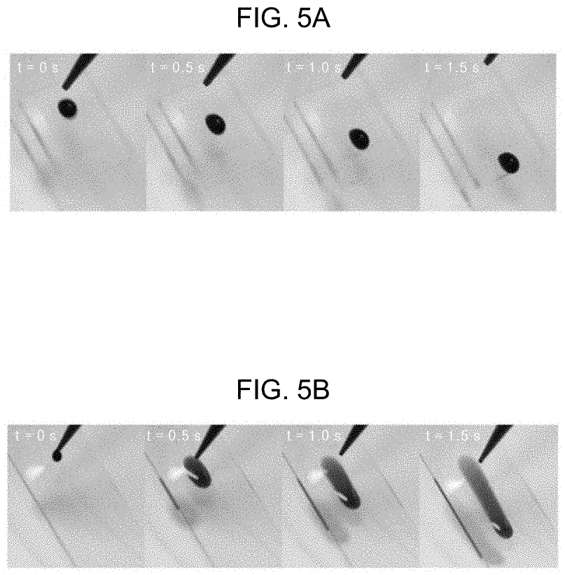

FIGS. 5A and 5B shows images of blood spreading on lenses according to an experimental example of the present invention and a comparative example depending on formation of an SAM coating layer.

FIG. 5A shows blood spreading on the lens according to Experimental Example 6 after forming the SAM coating layer on the lens having a nanometer-scale roughness. As a result, it may be confirmed that blood did not spread on the lens but rolled off the surface over time in the same shape as a dropped shape.

On the contrary, FIG. 5B shows blood spreading on the lens according to Comparative Example 1 which was not surface-treated and did not include the SAM coating layer, and it was confirmed that blood started to spread on the surface of the lens at 0.5 seconds after dropping the blood and stick to the surface of the lens while flowing thereon.

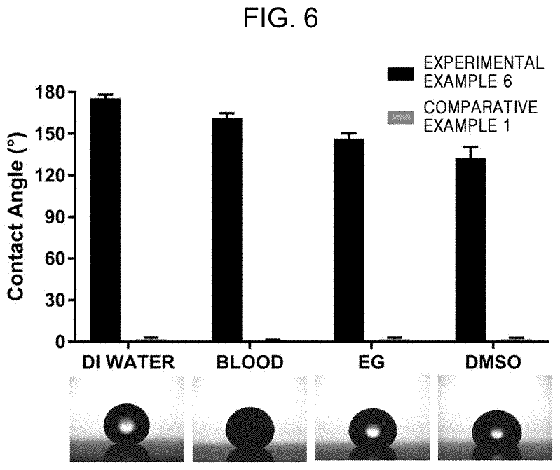

FIG. 6 is a graph illustrating contact angles of liquids with a surface of a lens according to an experimental example of the present invention depending on formation of an SAM coating layer on the surface of the lens.

Referring to FIG. 6, in the case of Comparative Example 1, it was confirmed that contact angles of different liquids such as distilled water, blood, and ethylene glycol (EG) with the lens converged on 0°. This indicates that the surface of the lens according to Comparative Example 1 has very high hydrophilicity. On the contrary, in the case of the lens according to Experimental Example 6 on which the SAM coating layer was formed, the liquids had very high contact angles of equal to or greater than 130° with the lens. This means that the surface of the lens according to Experimental Example 6 had super water-repellent properties.

Referring to FIGS. 5A, 5B and 6, it may be confirmed that the surface of the lens is hydrophobicized by surface treatment using the laser beam and SAM coating, thereby exhibiting super water-repellent properties.

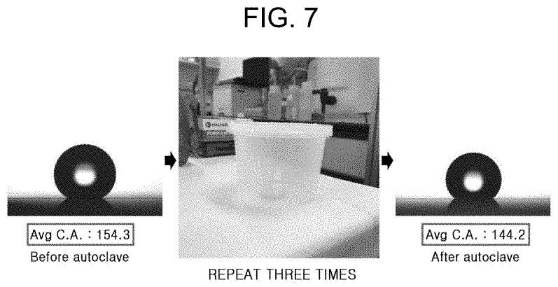

FIG. 7 shows a change in contact angle of a lens according to an experimental example of the present invention by sterilization and washing.

Medical devices used in the human body need to be sterilized before use to prevent contamination. Since the surface-treated lens according to the present invention is used in endoscopy, it was tested whether the SAM coating layer was maintained on the surface of the lens even under high-temperature, high-pressure sterilization conditions. To identify this, the lens according to Experimental Example 6 was subjected three times to high-temperature, high-pressure treatment using an autoclave, and a change in contact angle thereof was measured.

As a result, the measured contact angle with the lens was 154.3° before sterilization, and the contact angle was changed to 144.2° after repeating the treatment using the autoclave three times, indicating that the contact angle was maintained without a considerable change. Thus, it may be seen that the coating layer withstands high temperature and high pressure and the lens may be repeatedly used since the coating layer of the lens is maintained after repeating sterilization and washing.

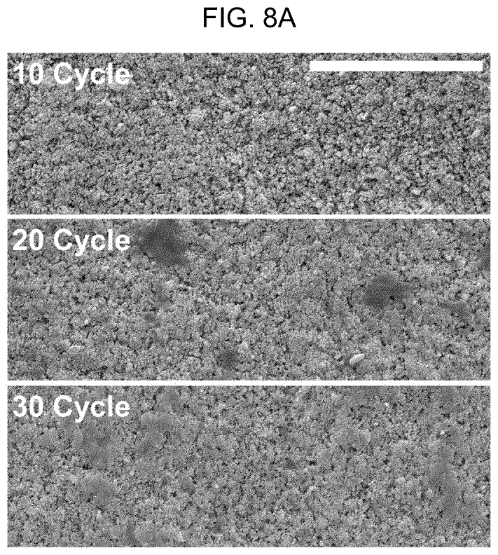

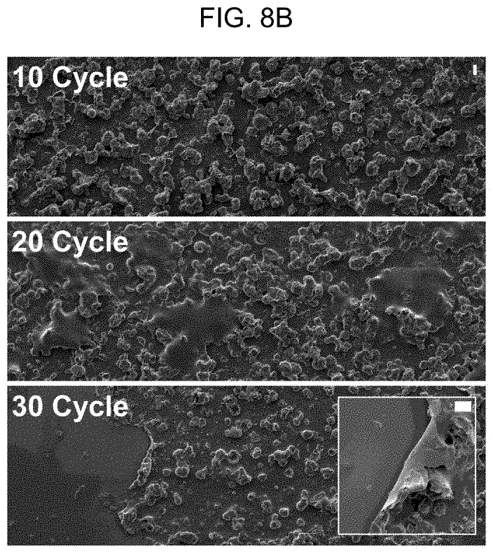

In order to identify adhesive strength of the SAM coating layer formed on the lens according to Experimental Example 6, a taping test was performed on the lenses according to Experimental Example 6 and Comparative Example 2, and the results are shown in FIGS. 8A and 8B.

For the taping test, a tape was completely attached to the surfaces of the lenses using a 4 kg-roller and detached, and this process was repeated. Changes of surfaces according to the number of repetitions were identified.

FIG. 8A shows results of the lens according to Experimental Example 6 after the taping test, and it was confirmed that the coating layer was maintained although the coating was partially pressed as the number of repetitions increased. FIG. 8B shows results of the lens according to Comparative Example 2 after the taping test, and it was confirmed that a larger portion of the coating layer was torn off as the number of taping tests increases. Thus, it may be seen that the SAM coating layer has far higher adhesive strength than that of conventional coating layers.

3. Applying Lubricant Layer

Various lubricant layers were formed on the SAM coating layer of the lens according to Experimental Example 6 and the lenses including the lubricant layers are referred to as Experimental Examples 7 to 10 according to the type of the lubricant. Table 2 below shows details of Experimental Examples 6 to 10.

TABLE 2

| No. | SAM coating | Lubricant application |
|---|---|---|
| Experimental Example 6 | FDTS coating | — |
| Experimental Example 7 | FDTS coating | GPL 103 |
| Experimental Example 8 | FDTS coating | GPL 101 |
| Experimental Example 9 | FDTS coating | PFD |
| Experimental Example 10 | FDTS coating | GPL 100 |

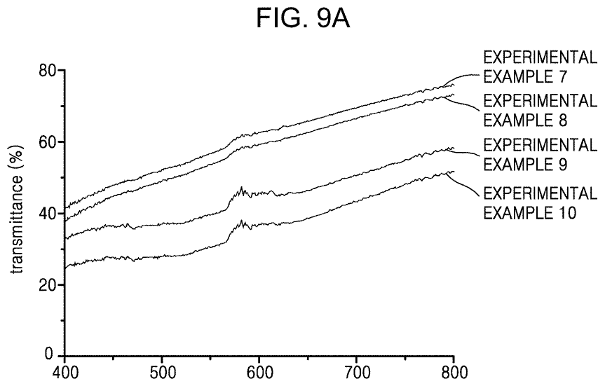

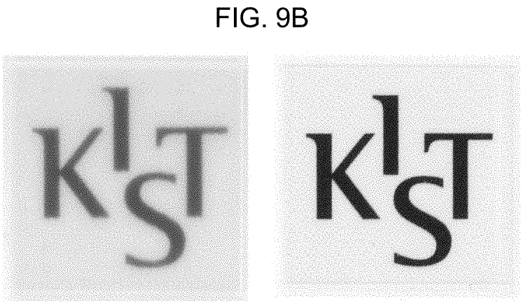

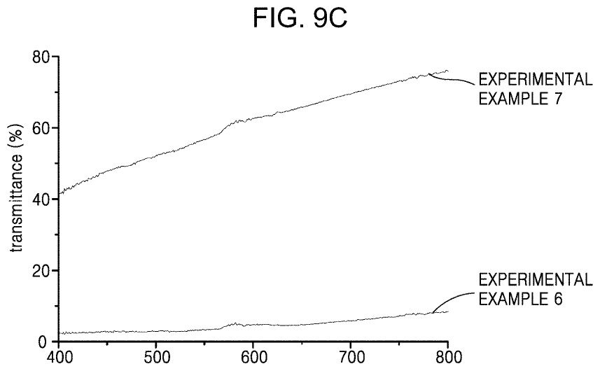

FIGS. 9A, 9B and 9C show comparison results of transmittance of the lenses according to experimental embodiments of the present invention and comparative examples depending on application of a lubricant after forming the microstructural layer and the SAM coating layer.

First, FIG. 9A shows comparison results of transmittances of the lenses according to the type of the lubricant and it may be confirmed that all lenses have a transmittance of 40% or more at 800 nm. Among them, it can be seen that a transmittance of 70% or more is obtained when GPL 103 of Experimental Example 7 is used.

FIG. 9B shows images of the lenses according to Experimental Examples 6 and 7 before measuring transmittance, and a considerable difference in transmittance was observed by application of the lubricant. Also, transmittances thereof were measured and shown in FIG. 9C. As a result, it can be seen that the lens according to Experimental Example 6 has a transmittance of less than 10% indicating about one seventh of that of the lens according to Experimental Example 7.

Therefore, it was confirmed that the lens had excellent super water-repellent properties by treating the surface of the lens with fine protrusions and forming the SAM coating layer thereon. Furthermore, the lens may be efficiently used for an endoscope due to excellent adhesive strength of the SAM coating layer. Also, it was confirmed that when the lubricant is applied to the upper surface of the SAM coating layer of the lens, transmittance was increased.

As described above, according to the manufacturing method of the present invention, problems of conventional lenses for medical devices including contamination of lens surfaces and low durability of coating layers may be efficiently overcome. In addition, since transmittance of the lens is increased by additionally forming the lubricant layer, a clear view may be stably obtained during surgery. Also, the lens according to an embodiment of the present invention is harmless to and does not irritate the human body without producing unpleasant odor. However, these effects are exemplary, and the scope of the present invention is not limited thereby.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A lens for a medical device, the lens comprising:
a microstructural layer on a surface of the lens; and
a coating layer on the microstructural layer,
wherein the coating layer is made of a self-assembled monolayer (SAM).

2. The lens of claim 1, wherein the coating layer comprises at least one selected from (heptadecafluoro-1,1,2,2,-tetrahydrodecyl)trichlorosilane (FDTS), trichloro(1H,1H,2H,2H-perfluorooctyl)silane (PFOTS), triethoxy(1H,1H,2H,2H-perfluoro-1-octyl)silane (POTS), and 1H,1H,2H,2H-perfluorodecyltrichlorosilane (FDDTS).

3. The lens of claim 1, further comprising a lubricant layer on the coating layer.

4. The lens of claim 3, wherein the lubricant layer comprises fluorinated synthetic oil or silicone oil.

5. The lens of claim 4, wherein the fluorinated synthetic oil comprises perfluoropolyether (PFPE) or perfluorodecalin (PFD).

6. The lens of claim 4, wherein the lubricant layer has a refractive index of 1.3 to 1.7.

7. The lens of claim 1, wherein the microstructural layer comprises fine protrusions each having a size of 0.5 μm to 0.8 μm.

8. The lens of claim 1, wherein the microstructural layer comprises fine protrusions each having a size of 90 nm to 175 nm.

9. A method of manufacturing a lens for a medical device, the method comprising:
forming a microstructural layer on a surface of the lens; and
forming a coating layer on the surface on which the microstructure is formed,
wherein the coating layer is formed of a self-assembled monolayer (SAM).

10. The method of claim 8, wherein the forming of the microstructural layer comprises at least one process selected from emitting a laser beam or electron beam to the surface of the lens, chemically etching the surface of the lens, and injecting-molding using a mold having a microstructure formed on the surface.

11. The method of claim 9, wherein the microstructure formed on the surface of the mold is formed by a laser beam.

12. The method of claim 9, wherein the forming of the microstructural layer is performed by forming a pattern comprising fine protrusions by emitting a laser beam to the surface of the lens.

13. The method of claim 12, wherein the pattern is formed by adjusting a transverse pulse overlap rate and a longitudinal pulse overlap rate of the laser beam.

14. The method of claim 13, wherein the sizes of the fine protrusions are adjusted within the range of 0.5 μm to 0.8 μm by controlling the transverse pulse overlap rate to be equal to or more than 0% and equal to or less than 50% and the longitudinal pulse overlap rate to be equal to or more than 50% and equal to or less than 75%.

15. The method of claim 13, wherein the sizes of the fine protrusions are adjusted within the range of 90 nm to 175 nm by controlling the transverse pulse overlap rate to be more than 50% and less than 99.9% and the longitudinal pulse overlap rate to be more than 75% and less than 99.9%.

16. The method of claim 9, wherein the SAM comprises at least one selected from (heptadecafluoro-1,1,2,2,-tetrahydrodecyl)trichlorosilane (FDTS), trichloro(1H,1H,2H,2H-perfluorooctyl)silane (PFOTS), triethoxy(1H,1H,2H,2H-perfluoro-1-octyl)silane (POTS), and 1H,1H,2H,2H-perfluorodecyltrichlorosilane (FDDTS).

17. The method of claim 9, further comprising applying a lubricant on the surface of the lens on which) the coating layer is formed after the forming of the coating layer.

18. The method of claim 17, wherein the lubricant comprises fluorinated synthetic oil or silicone oil.

19. A method of increasing transmittance of a lens for a medical device, the lens comprising a microstructural layer formed on a surface and a coating layer formed on the microstructural layer as a self-assembled monolayer (SAM), the method comprising:
applying a lubricant having a refractive index corresponding to 80% to 120% of a refractive index of the lens to the coating layer.
